(19) United States
(12) Patent Application Publication
(10) Pub. No.: US 2014/0163587 A1
(43) Pub. Date: Jun. 12, 2014

(54) NERVE GUIDE CONDUIT

(71) Applicant: INDUSTRIAL TECHNOLOGY RESEARCH INSTITUTE, (US)

(72) Inventors: Ming-Chia Yang, Taipei City (TW); Chun-Jen Liao, Taipei City (TW); Wen-Hsiang Chang, Hsinchu City (TW); Wei-Ju Liao, Taichung City (TW); Ming-Hong Chen, Taipei City (TW)

(73) Assignee: INDUSTRIAL TECHNOLOGY RESEARCH INSTITUTE, HSINCHU (TW)

(21) Appl. No.: 13/762,505

(22) Filed: Feb. 8, 2013

(30) Foreign Application Priority Data
Dec. 10, 2012 (TW) 101223906

(51) Int. Cl. A61B 17/11

(52) U.S. Cl. CPC A61B 17/1128
USPC 606/152

(57) ABSTRACT

A nerve guide conduit comprising a first open cover, a second open cover and a connecting portion is provided. The first open cover has a first guiding recess, a first terminal surface and a second terminal surface opposite to the first terminal surface. The first guiding recess is extended to the second terminal surface from the first terminal surface. The second open cover has a second guiding recess, a third terminal surface and a fourth terminal surface opposite to the third terminal surface. The second guiding recess is extended to the fourth terminal surface from the third terminal surface. When the first open cover covers the second open cover, the first guiding recess and the second guiding recess together provide a nerve receiving space. The connecting portion connects the first open cover and the second open cover.

### CROSS REFERENCE TO RELATED APPLICATIONS

This application claims the benefit of Taiwan application Serial No. 101223906, filed Dec. 10, 2012, the disclosure of which is incorporated by reference herein in its entirety.

### TECHNICAL FIELD

The disclosure relates in general to a nerve guide conduit, and more particularly to a nerve guide conduit with a guiding recess.

### BACKGROUND

Peripheral nerve injury (PNI) is a commonly seen nerve injury in clinical practice, and is mostly caused by accidents which make a peripheral nerve compressed or severed. Some nerve injuries are not permanent (caused by compression), and will rehabilitate after a period of rest. Severe permanent peripheral nerve injuries normally require an interventional treatment such as neurosurgical procedure to rehabilitate. The regeneration of injured nerve is normally subjected to biological, chemical, and physical obstacle. The biological obstacle refers to the repaired nerve failing to form neuromuscular junctions with the muscle and incapacitating the recovery of the muscle driven by the nerve. The chemical obstacle refers to the protein message transmission of an organism suppressing the proliferation of the nerve. The physical obstacle refers to the connective tissue barrier, arising from the clustering of stellate cells or Schwann cells, occurs to the nerve fracture and incapacitates the growth of the nerve.

To cure the injured nerve, a closed type cylindrical conduit is normally in generally known technology to join the fracture of the nerve to two terminals of the cylindrical conduit respectively, such that the nerve can gradually recover through the guide of the pilot hole in the cylindrical conduit. However, the closed type cylindrical conduit is only applicable to the completely broken nerve, and has very limited application.

### SUMMARY

According to one embodiment, a nerve guide conduit is provided. The nerve guide conduit comprises a first open cover, a second open cover and a connecting portion. The first open cover has a first guiding recess, a first terminal surface and a second terminal surface opposite to the first terminal surface. The first guiding recess is extended to the second terminal surface from the first terminal surface. The second open cover has a second guiding recess, a third terminal surface and a fourth terminal surface opposite to the third terminal surface. The second guiding recess is extended to the fourth terminal surface from the third terminal surface. When the first open cover covers the second open cover, the first guiding recess and the second guiding recess together provide a nerve receiving space. The connecting portion connects the first open cover and the second open cover.

### BRIEF DESCRIPTION OF THE DRAWINGS

FIG. 1 shows an expansion diagram of a nerve guide conduit according to an embodiment of the disclosure;

FIG. 2 shows an appearance diagram of nerve received in a first open cover of FIG. 1;

FIG. 3 shows a cross-sectional view along direction 3-3′ of FIG. 1;

FIG. 4 shows a cross-sectional view along direction 4-4′ of FIG. 1;

FIG. 5 shows an expansion diagram of a nerve guide conduit according to another embodiment of the disclosure;

FIG. 6 shows a nerve guide conduit in a closed state according to another embodiment of the disclosure;

FIG. 7 shows an expansion diagram of a nerve guide conduit according to another embodiment of the disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

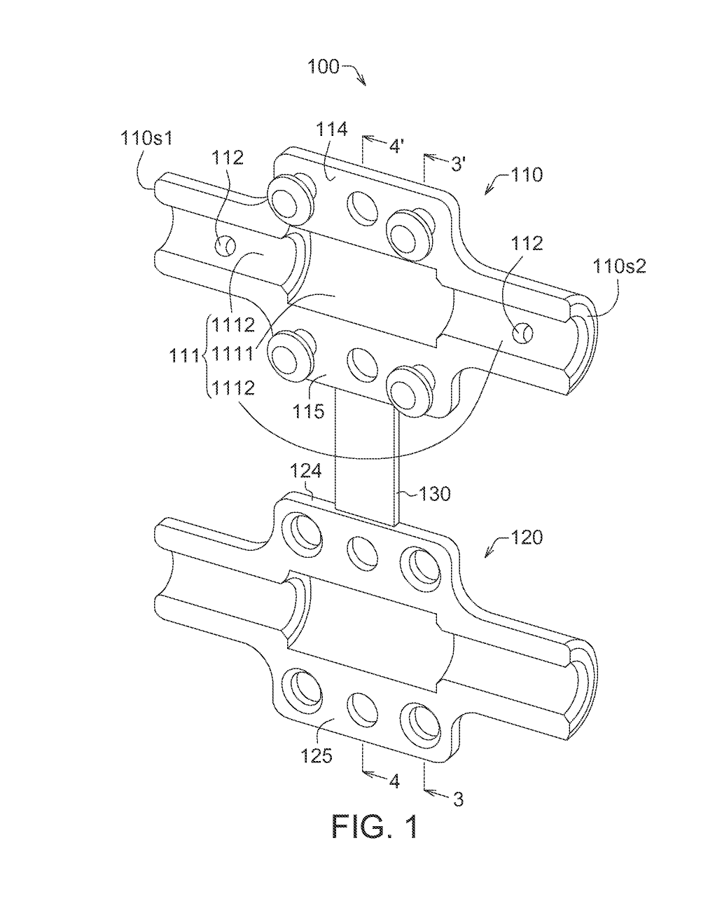

Referring to FIG. 1, an expansion diagram of a nerve guide conduit according to an embodiment of the disclosure is shown. The nerve guide conduit 100 comprises a first open cover 110, a second open cover 120 and a connecting portion 130.

The first open cover 110 has a first guiding recess 111, a first terminal surface 110s1 and a second terminal surface 110s2 opposite to the first terminal surface 110s1. The first guiding recess 111 is extended to the second terminal surface 110s2 from the first terminal surface 110s1. The first guiding recess 111 comprises a first drug-receiving recess 1111 and two first sub-recesses 1112, wherein two terminals of the first drug-receiving recess 1111 respectively connect two first sub-recesses 1112 for receiving nerve therapy drug, such as nerve growth promoting hydrogel, particles and film. The drug-receiving slot has many holes or storage tanks for receiving a gel or a film containing enzymes or growth factors which can be used for guiding nerve cell conglutination and growth. Examples of enzymes comprise liberase, collagenase, hyaluronidase, hyparin. Examples of growth factors comprise nerve growth factor, brain-derived neurotrophic factor, ciliary neurotrophic factor, fibroblast growth factors and glial cell line-derived neurotrophic factor. Enzymes can be used for treating the soft tissue surface of the nerve. After enzymes treatment, the extracellular matrix of the tissue will become loose and result in cellular dissociation, which makes it easier for cells to obtain sufficient nutrient and proliferate. The proliferated cells will secrete a larger amount of extracellular matrixes, which benefit the repair and fusion between tissues. The present method for treating injured tissue confines the site of enzymes treatment to the injury site so that the physiological conditions of peripheral tissue will not be affected.

Two first sub-recesses 1112 are extended to the first terminal surface 110s1 and the second terminal surface 110s2 respectively. The thickness of the first open cover 110 is between 50˜3000 nanometers.

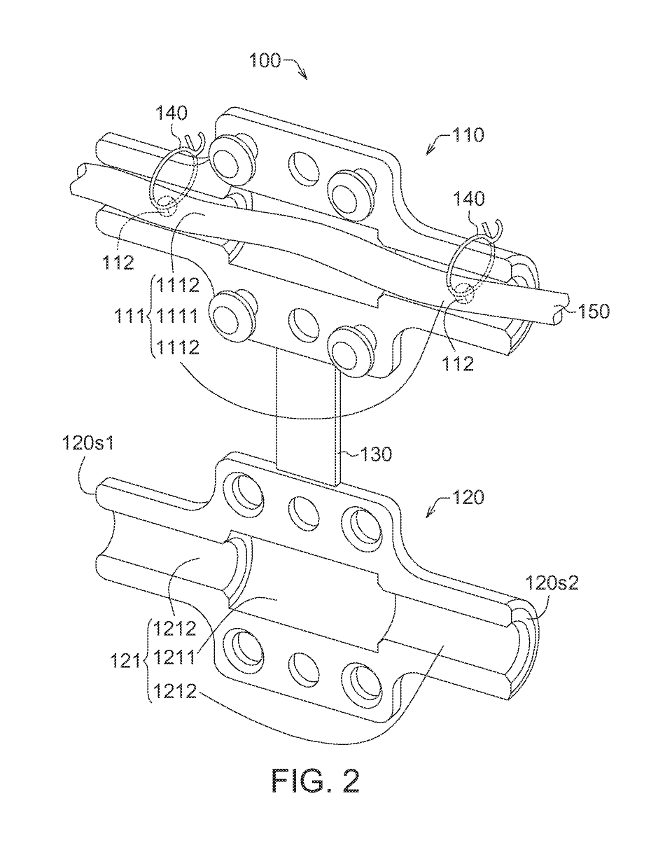

Referring to FIG. 2, an appearance diagram of nerve received in a first open cover of FIG. 1 is shown. The first open cover 110 has at least one suture hole 112 extended to an outer wall of the first open cover 110 from an inner wall of the first sub-recess 1112. The suture 140 passes through the suture hole 112 to fix the nerve 150 in the first sub-recess 1112 to avoid the nerve 150 coming off the first open cover 110. In the present example, the quantity of suture holes 112 is exemplified by two suture holes 112 respectively formed in two first sub-recesses 1112. However, the quantity of suture holes 112 can be exemplified by one suture hole 112 or more than two suture holes 112 formed in at least one of the first drug-receiving recess 1111 and the first sub-recess 1112. In an example, the inner diameter of suture holes 112 is smaller than 3 micrometers or other suitable measurement. The disclosure does not impose further restrictions on the inner diameter of suture holes 112 as long as the measurement goes with the suture 140.

The first open cover 110 is flexible and can be formed by biodegradable or compatible material, including biodegradable polyester such as PLA, PGA, PCL and their heterometric molecular polymer, amino acid, silicone and their derivatives, and natural biodegradable polymers, wherein the natural biodegradable polymers are such as chitosan, collagen, alginate esters and their derivatives. In an example, the first open cover 110 is formed by material such as silicone, chitosan, poly lactic acid, PGA (poly glycolic acid), poly (glycolide co-lactide); PLGA, collage, poly hydroxy acids, alginate, polyamide and their derivatives. In another example, the first open cover 110 is formed by a porous material capable of receiving or absorbing nerve therapy drug. In another example, the first open cover 110 can be formed by a hard material.

The second open cover 120 has a second guiding recess 121, a third terminal surface 120s1 and a fourth terminal surface 120s2 opposite to the third terminal surface 120s1. The second guiding recess 121 is extended to the fourth terminal surface 120s2 from the third terminal surface 120s1. When the first open cover 110 covers the second open cover 120, the first guiding recess 111 and the second guiding recess 121 together provide a nerve receiving space for receiving a completely broken or an injured but unbroken nerve 150. The second guiding recess 121 comprises a second drug-receiving recess 1211 and two second sub-recesses 1212, wherein two terminals of the second drug-receiving recess 1211 respectively are connected to two second sub-recesses 1212 for receiving nerve therapy drug, and the two second sub-recesses 1212 respectively are extended to the third terminal surface 120s1 and the fourth terminal surface 120s2.

In an example, the suture hole 112 of FIG. 1 can also be formed on the second open cover 120 to generate similar nerve fixing function. Under such design, the suture hole 112 of the first open cover 110 can be selectively omitted.

The material of the second open cover 120 is similar to that of the first open cover 110, and the similarities are not repeated here. In another example, the second open cover 120 can be made from a porous material capable of receiving or absorbing nerve therapy drug. The second open cover 120 and the first open cover 110 can be formed by the same or different materials.

The connecting portion 130 connects the first open cover 110 and the second open cover 120. The material of the connecting portion 130 can be similar to that of the first open cover 110 and/or the second open cover 120, and the similarities are not repeated here. In the present example, the first open cover 110, the second open cover 120 and the connecting portion 130 are integrally formed in one piece.

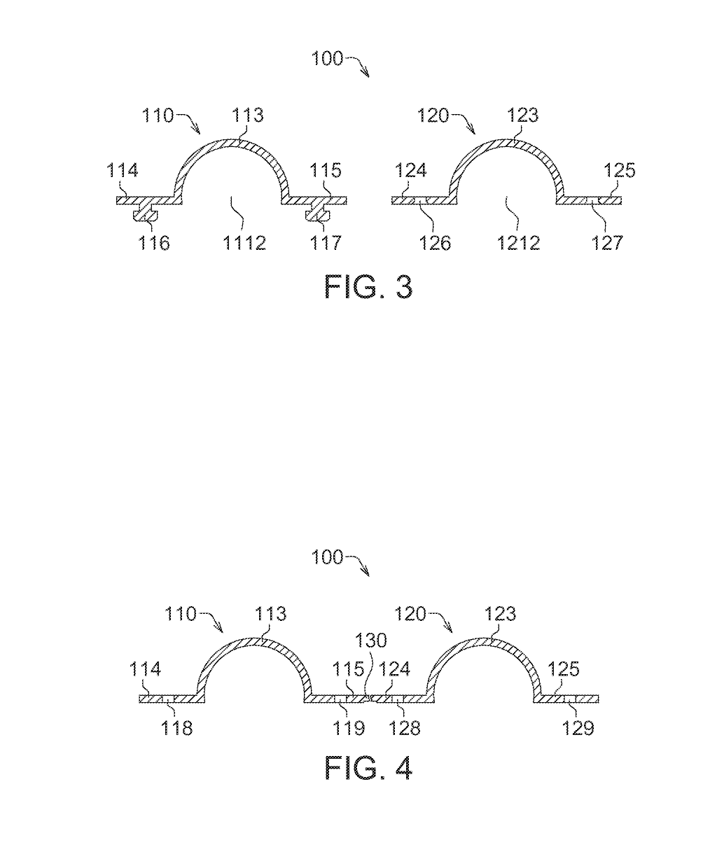

Referring to FIG. 3, FIG. 1 a cross-sectional view along direction 3-3′ of FIG. 1 is shown. The first open cover 110 comprises a first tube 113, a first flank 114 and a second flank 115, wherein the first flank 114 and the second flank 115 are connected to two opposite sides of the first tube 113. The first tube 113 is an open type tube, such that the first tube 113 exposes the first drug-receiving recess 1111. The cross-sectional contour of the first tube 113 is such as a portion of a circle, a portion of an ellipse or a portion of a polygon.

The second open cover 120 comprises a second tube 123, a third flank 124 and a fourth flank 125, wherein the third flank 124 and the fourth flank 125 are connected to two opposite sides of the second tube 123. The second tube 123 is an open type tube, such that the second tube 123 exposes the second drug-receiving recess 1211. The cross-sectional contour of second tube 123 is such as a portion of a circle, a portion of an ellipse or a portion of a polygon. Since the third flank 124 and the second flank 115 are adjacent to each other, the second flank 115 is fixed in the third flank 124 and the first flank 114 is fixed in the fourth flank 125 when the first open cover 110 covers the second open cover 120.

The first open cover 110 further comprises at least one first engaging portion 116 and at least one second engaging portion 117 respectively are disposed on the first flank 114 and the second flank 115, wherein the first engaging portion 116 and the second engaging portion 117 and the first drug-receiving recess 1111 are located on the same side of the first open cover 110. The second open cover 120 comprises at least one third engaging portion 126 and at least one fourth engaging portion 127 respectively disposed on the third flank 124 and the fourth flank 125. When the first open cover 110 covers the second open cover 120, the first engaging portion 116 is engaged with the fourth engaging portion 127, and the second engaging portion 117 is fixed in the third engaging portion 126. In the present example, both the first engaging portion 116 and the second engaging portion 117 are a protrusion, and both the third engaging portion 126 and the fourth engaging portion 127 are an indentation such as a recess or a through hole, wherein the outer diameter of the protrusion is larger than the inner diameter of the indentation, such that the protrusion can be firmly engaged in the indentation for firmly fixing the nerve 150 (FIG. 2) in the nerve guide conduit 100. In another example, the first engaging portion 116 and the second engaging portion 117 can both be an indentation, and the third engaging portion 126 and the fourth engaging portion 127 can both be a protrusion. In another example, one of the first engaging portion 116 and the third engaging portion 126 can be a protrusion, and the other of the second engaging portion 117 and the fourth engaging portion 127 can be an indentation; Alternatively, one of the first engaging portion 116 and the third engaging portion 126 can be an indentation, and the other of the second engaging portion 117 and the fourth engaging portion 127 can be a protrusion.

Referring to FIG. 4, a cross-sectional view along direction 4-4′ of FIG. 1 is shown. The first open cover 110 further has a first through hole 118 and a second through hole 119 respectively passing through the first flank 114 and the second flank 115. The second open cover 120 further has a third through hole 128 and a fourth through hole 129respectively passing through the third flank 124 and the fourth flank 125. The second through hole 119 and the third through hole 128 are adjacent to each other, such that when the first open cover 110 covers the second open cover 120, another suture 140 can pass through the second through hole 119 and the third through hole 128 to fix the first open cover 110 and the second open cover 120, and another suture 140 can pass through the first through hole 118 and the fourth through hole 129 to fix the first open cover 110 and the second open cover 120. In another example, the second through hole 119 and the third through hole 128 can be omitted. Alternatively, the first through hole 118 and the fourth through hole 129 can be omitted.

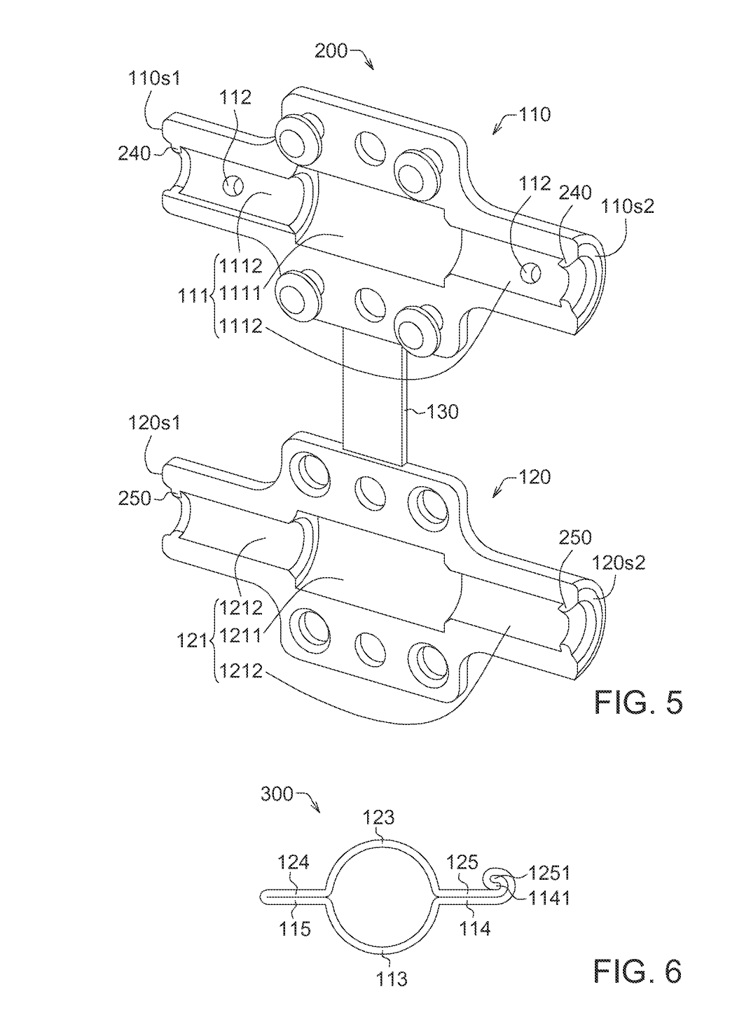

Referring to FIG. 5, an expansion diagram of a nerve guide conduit according to another embodiment of the disclosure is shown. The nerve guide conduit 200 comprises a first open cover 110, a second open cover 120, a connecting portion 130, two first protrusions 240 and two second protrusions 250.

The first protrusion 240 is disposed on an inner wall of the first guiding recess 111 and adjacent to the first terminal surface 110s1 and the second terminal surface 110s2 respectively for engaging the nerve disposed in the first guiding recess 111 (not illustrated). Likewise, the second protrusion 250 is disposed on an inner wall of the second guiding recess 121 and adjacent to the third terminal surface 120s1 and the fourth terminal surface 120s2 respectively for engaging the nerve (not illustrated) disposed on the second guiding recess 121.

Referring to FIG. 6, a nerve guide conduit in a closed state according to another embodiment of the disclosure is shown. The nerve guide conduit 300 comprises a first open cover 110, a second open cover 120 and a connecting portion 130.

The first open cover 110 comprises a first flank 114 and a second flank 115. The second open cover 120 comprises a third flank 124 and a fourth flank 125. The free terminal of the first flank 114 is the first hook 1141, and the free terminal of the fourth flank 125 is the second hook 1251. When the first open cover 110 covers the second open cover 120, the first hook 1141 can be engaged with the second hook 1251 to avoid the first open cover 110 coming off the second open cover 120.

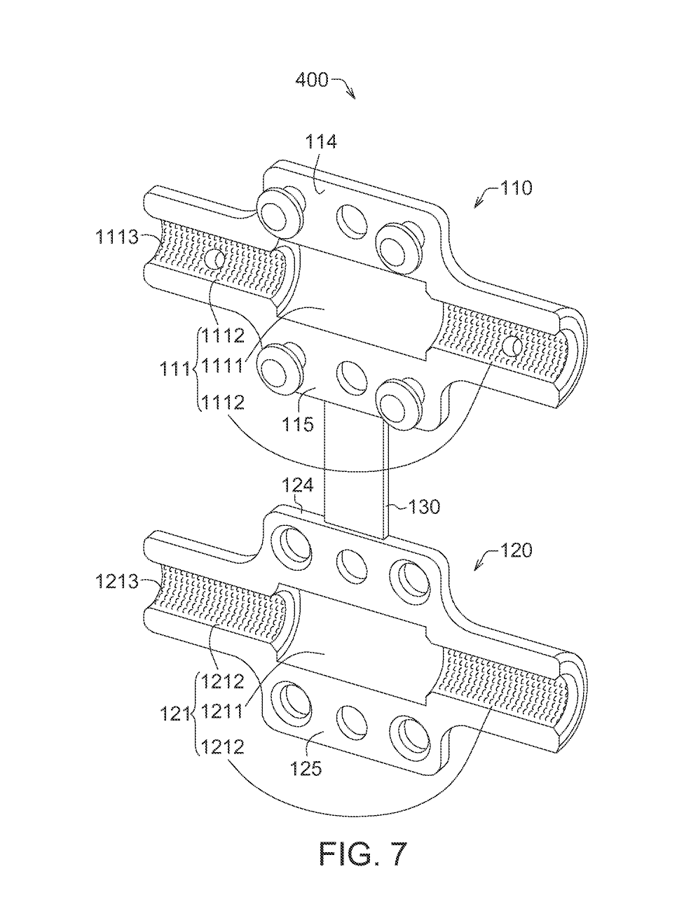

Referring to FIG. 7, an expansion diagram of a nerve guide conduit according to another embodiment of the disclosure is shown. The nerve guide conduit 400 comprises a first open cover 110, a second open cover 120 and a connecting portion 130.

The first open cover 110 has a first guiding recess 111, which comprises a first drug-receiving recess 1111 and two first sub-recesses 1112, wherein two terminals of the first drug-receiving recess 1111 respectively connect two first sub-recesses 1112. The second open cover 120 has a second guiding recess 121, which comprises a second drug-receiving recess 1211 and two second sub-recesses 1212, wherein two terminals of the second drug-receiving recess 1211 respectively are connected to two second sub-recess 1212.

The first open cover 110 has a rough structure 1113 formed on the inner wall of the first sub-recess 1112. When the nerve (not illustrated) contacts the rough structure 1113 of the first sub-recess 1112, the rough structure 1113 provides a resisting force (frictional force) to avoid the nerve easily coming off the nerve guide conduit 400. In another example, the rough structure 1113 can also be formed on the inner wall of the first drug-receiving recess 1111. The measurement of the rough structure 1113 is between nanometer and micrometer. Likewise, the second open cover 120 has a rough structure 1213 formed on the inner wall of the second sub-recess 1212. When the nerve (not illustrated) contacts the rough structure 1213 of the second sub-recess 1212, the rough structure 1213 provides a resisting force (frictional force) to avoid the nerve easily coming off the nerve guide conduit 400. In another example, the rough structure 1213 can also be formed on the inner wall of the first drug-receiving recess 1111. Besides, the measurement of the rough structure 1213 is between nanometer and micrometer.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A nerve guide conduit, comprising:
a first open cover having a first guiding recess and a first terminal surface and a second terminal surface opposite to the first terminal surface, wherein the first guiding recess is extended to the second terminal surface from the first terminal surface;
a second open cover having a second guiding recess and a third terminal surface and a fourth terminal surface opposite to the third terminal surface, wherein the second guiding recess is extended to the fourth terminal surface from the third terminal surface, and when the first open cover covers the second open cover, the first guiding recess and the second guiding recess together provide a nerve receiving space; and
a connecting portion connecting the first open cover and the second open cover.

2. The nerve guide conduit according to claim 1, wherein the first open cover, the second open cover and the connecting portion are flexible.

3. The nerve guide conduit according to claim 1, wherein the first guiding recess comprises a first drug-receiving recess used for receiving nerve therapy drug.

4. The nerve guide conduit according to claim 1, wherein the second guiding recess comprises a second drug-receiving recess used for receiving nerve therapy drug.

5. The nerve guide conduit according to claim 1, wherein the first guiding recess comprises a first drug-receiving recess and two first sub-recesses, and two terminals of the first drug-receiving recess respectively connect the two first sub-recesses.

6. The nerve guide conduit according to claim 1, wherein the second guiding recess comprises a second drug-receiving recess and two second sub-recesses, and two terminals of the second drug-receiving recess respectively connect the two second sub-recesses.

7. The nerve guide conduit according to claim 1, wherein the first open cover has a rough structure formed on an inner wall of the first guiding recess.

8. The nerve guide conduit according to claim 1, wherein the second open cover has a rough structure formed on an inner wall of the second guiding recess.

9. The nerve guide conduit according to claim 1, wherein the first open cover and the second open cover are formed by a porous material.

10. The nerve guide conduit according to claim 1, wherein the first open cover comprises a first tube, a first flank and a second flank, the first flank and the second flank are connected to two opposite sides of the first tube, the second open cover comprises a second tube, a third flank and a fourth flank, the third flank and the fourth flank are connected to two opposite sides of the second tube, the second flank and the third flank are adjacent to each other, and when the first open cover covers the second open cover, the first flank is fixed in the fourth flank, and the second flank is fixed in the third flank.

11. The nerve guide conduit according to claim 10, wherein the first open cover comprises a first engaging portion and a second engaging portion, the first engaging portion and the second engaging portion are respectively disposed on the first flank and the second flank; the second open cover comprises a third engaging portion and a fourth engaging portion, the third engaging portion and the fourth engaging portion are respectively disposed on the third flank and the fourth flank, and when the first open cover covers the second open cover, the first engaging portion is engaged with the fourth engaging portion, and the second engaging portion is fixed in the third engaging portion.

12. The nerve guide conduit according to claim 11, wherein one of the first engaging portion and the third engaging portion is a protrusion, and the other of the first engaging portion and the third engaging portion is an indentation, one of the second engaging portion and the fourth engaging portion is a protrusion, and the other of the second engaging portion and the fourth engaging portion is an indentation.

13. The nerve guide conduit according to claim 10, wherein the first open cover has a first through hole and a second through hole, the first through hole and the second through hole respectively pass through the first flank and the second flank, and when the first open cover covers the second open cover, a suture passes through the second through hole and the third through hole to fix the first open cover and the second open cover.

14. The nerve guide conduit according to claim 13, wherein the second open cover further has a third through hole and a fourth through hole, the third through hole and the fourth through hole respectively pass through the third flank and the fourth flank, the second through hole and the third through hole are adjacent to each other, such that when the first open cover covers the second open cover, another suture passes through the first through hole and the fourth through hole to fix the first open cover and the second open cover.

15. The nerve guide conduit according to claim 10, wherein a free terminal of the first flank is a first hook, a free terminal of the fourth flank is a second hook, and the first hook is engaged with the second hook.

16. The nerve guide conduit according to claim 1, wherein the first open cover has a suture hole extended to an outer wall of the first open cover from an inner wall of the first guiding recess.

17. The nerve guide conduit according to claim 16, wherein the first guiding recess comprises a first drug-receiving recess and a first sub-recess, the first sub-recess is extended to the first drug-receiving recess from the first terminal surface, and the suture hole is extended to an outer wall of the first open cover from an inner wall of the first sub-recess.

18. The nerve guide conduit according to claim 1, wherein the first guiding recess comprises a first drug-receiving recess and two first sub-recesses, the two first sub-recess are respectively extended to the first drug-receiving recess from the first terminal surface and the second terminal surface, the first open cover has two suture holes respectively extended to an outer wall of the first open cover from an inner wall of the two first sub-recesses.

19. The nerve guide conduit according to claim 1, further comprising:
two first protrusions disposed on an inner wall of the first guiding recess adjacent to the first terminal surface and the second terminal surface respectively.

20. The nerve guide conduit according to claim 1, further comprising:
two second protrusions disposed on an inner wall of the second guiding recess adjacent to the third terminal surface and the fourth terminal surface respectively.
